(19) United States
(12) Patent Application Publication
(10) Pub. No.: US 2008/0026441 A1
(43) Pub. Date: Jan. 31, 2008

(54) Production of probiotic bacteria using maple sap

(76) Inventors: Diane Fournier, Montreal (CA); Jalal Hawari, St. Laurent (CA); Armelle Cochu, Montreal (CA)

Correspondence Address: NATIONAL RESEARCH COUNCIL OF CANADA;1200 MONTREAL ROAD, BLDG M-58, ROOM EG12, OTTAWA, ONTARIO, K1A 0R6, CA

(21) Appl. No.: 11/878,293

(22) Filed: Jul. 23, 2007

Related U.S. Application Data
(60) Provisional application No. 60/833,277, filed on Jul. 26, 2006.

(51) Int. Cl. C12P 7/56; C12N 1/20

(52) U.S. Cl. 435/139; 435/252.1; 435/252.9

(57) ABSTRACT

Use of maple sap, down-graded maple syrup or mixture thereof as a carbon and energy source for the production of probiotic bacteria (e.g. Lactobacilli spp.) unexpectedly leads to an improvement in growth of the probiotic bacteria and an improvement in the yield of products produced by the bacteria.

### CROSS-REFERENCE TO RELATED APPLICATIONS

This application claims the benefit of U.S. Provisional Patent Application Ser. No. 60/833,277 filed Jul. 26, 2006.

### FIELD OF THE INVENTION

The present invention relates to biotechnology, in particular to methods for producing probiotic bacteria and products of probiotic bacteria.

### BACKGROUND OF THE INVENTION

Maple syrup is one of the hallmark products of Canada. About 84% of the world's production of maple syrup is made in Canada and more than 93% of it originates from the province of Quebec. Since 1999, a dramatic saturation of the markets has led to a large inventory surplus. At the end of the 2004 season, the volume of bulk inventories exceeded 60 million pounds. The Fédération des Producteurs Acéricoles du Québec is addressing the surplus problem by exploring new markets.

Maple sap, the sap of the maple tree, can be considered as a ready-to-use, sugar-rich (mainly sucrose), renewable feedstock having a potential to sustain the growth of a large variety of microorganisms. In commonly owned U.S. patent application Ser. No. 11/715,944 field Mar. 9, 2007, the feasibility of growing large amounts of the polyhydroxyalcanoate-producing bacterium, Alcaligenes latus, was demonstrated.

According to the definition of the United Nations Food and Agricultural Organization and the World Health Organization, probiotics are “live microorganisms which, when administrated in adequate amounts, confer a health benefit on the host”. There is already an important market for probiotics as food supplements for the benefit of human and animal health. In a colloquium of the American Academy for Microbiology held in Baltimore in November 2005, participants with expertise in microbiology, medicine, nutrition, immunology, animal sciences and other relevant field listed the following examples where probiotics use benefit human and animal health: treating diarrhea caused by Rotavirus in children, treating irritable bowel syndrome, treating bladder cancer, treating urogenital infections, treating Clostridium difficile infections, and treating atopic eczema.

Prior art processes for the production of probiotics using various carbon and nitrogen sources, e.g. sugarcane, are well known and many companies already sell probiotic products. However, there remains a need in the art for improved methods of producing probiotic bacteria.

### SUMMARY OF THE INVENTION

Here we disclose the use of maple sap or down-graded maple syrup as a carbon and energy source for the production of probiotic bacteria. The use of maple sap or down-graded maple syrup unexpectedly leads to an improvement in growth of probiotic bacteria and an improvement in the yield of products produced by the bacteria.

Thus, there is provided a method of growing probiotic bacteria comprising contacting the probiotic bacteria with maple sap, down-graded maple syrup or a mixture thereof.

There is further provided a method of producing lactic acid comprising contacting probiotic Lactobacillus bacteria with maple sap, or down-graded maple syrup or a mixture thereof.

Probiotic bacteria are preferably bacteria, or mixtures of bacteria, of genus Lactobacillus, for example L. acidophilus, L. brevis, L. buchneri, L. casei, L. curvatus, L. delbrueckii, L. fermentum, L. helveticus, L. plantarum, L. reuteri, L. rhamnosus, L. sakei and L. salivarius. L. acidophilus, L. casei and L. helveticus are of particular note.

Maple sap-based culture medium in accordance with the present invention allows good growth of probiotic bacterial strains used in the production of commercial probiotic products. Maple sap-based medium surprisingly provides better growth yield than a sucrose-based medium. Growth yield on maple sap-based medium may be, for example, at least 1.5 times greater than growth yield on sucrose-based medium. In some embodiments, improvements in growth yield of at least 2 times greater, or at least 3 times greater, or at least 4 times greater may be realized. In other embodiments, improvement in growth yield may be 2 to 4 times greater.

Enhanced growth yield on maple sap-based medium may also lead to greater production of probiotic products, for example lactic acid, anti-oxidants and/or other biologically active components that trigger the growth of probiotic bacteria. In some embodiments, production of lactic acid (or lactate) may be at least 1.5 times greater, or at least 2 times greater, in a maple sap-based medium than in a sucrose-based medium.

Advantageously, the maple sap may be formulated into a medium containing growth supplements for the probiotic bacteria. Growth supplements may include, for example, a yeast extract, salts, a nitrogen source, vitamins or mixtures thereof. Salts include, for example, potassium dihydrogen phosphate (KH₂PO₄), dipotassium hydrogen phosphate (K₂HPO₄), manganese sulfate (MnSO₄), magnesium sulfate (MgSO₄), ferrous sulfate (FeSO₄), etc. Nitrogen sources include, for example, proteins from vegetal sources (e.g. soy, pea, etc.) or animal sources (e.g. milk proteins such as casein). Because there is an increasing demand for animal-free products in the food market (since the emergence of mad cow disease and increasing food allergies toward bovine proteins), the nitrogen source is preferably protein from vegetal sources. Vitamins include, for example, mevalonic acid, ascorbic acid, etc. Growth supplements may be used in any effective concentration, for example, in a range of from about 0.01% to about 100% of the concentration of the maple sap based on weight.

Incubation or fermentation of the bacteria in the medium may be conducted at a temperature in a range of from about 30° C. to about 45° C., preferably 36-43° C. Anaerobic or aerobic conditions may be used, preferably anaerobic conditions. Cultures may be static or agitated, preferably static.

Methods of the present invention are particularly useful for commercial scale production of probiotic bacteria or products from probiotic bacteria. Commercial scale production is preferably carried out in bioreactors where the bacteria are fermented in the maple sap-based medium.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 is a graph of OD₆₀₀ₙₘ vs. time (hours) depicting growth of probiotic bacteria in maple sap-based medium at 37° C. under static condition;

FIG. 2 is a graph of viable cell count (colony forming units (cfu)) at 0 hours and 16 hours for the growth of probiotic bacteria in maple sap-based medium at 37° C. under static condition;

FIG. 3 is a graph of viable cell count (colony forming units (cfu)) at 16 hours for the growth of probiotic bacteria in maple sap-based medium and sucrose-based medium at 37° C. under static condition; and,

FIG. 4 is a graph of lactate produced (ppm) after 16 hours for the growth of probiotic bacteria in maple sap-based medium and sucrose-based medium at 37° C. under static condition.

### DESCRIPTION OF PREFERRED EMBODIMENTS

### EXAMPLE 1

### Growth in Maple Sap-based Medium Supplemented With Soy Drink

Filtered-sterilized maple sap (50 mL) at pH 7.0 having about 16,000 ppm sucrose, about 400 ppm glucose and 400 ppm fructose was inoculated with 0.1 mL of commercial Bio-K+ product, which contains high amounts of probiotic fermentive bacteria, in this case, two lactobacilli, L. casei and L. acidophilus. Although maple sap is a good carbon source, it has a low carbon to nitrogen (C/N) ratio, therefore it was supplemented with either ammonium sulfate (2 mM) or a commercial soy drink, UHT, (30% (v/v) soy drink was added to 70% (v/v) maple sap) as a source of nitrogen. The cultures were incubated at 30° C. for two days under anaerobic conditions (in closed serum bottles with the headspace flushed with argon).

In the non-inoculated controls, no bacterial growth (i.e. no turbidity) was observed in any of the conditions mentioned. The non-supplemented maple sap and the maple sap supplemented with ammonium sulfate only led to poor bacterial growth. However, the aerobic culture composed of maple sap (70% (v/v)) and soy drink (30% (v/v)) led to the growth of an important bacterial concentration (approx. 4.55×10⁹ bacteria/mL using viable counts). After two days of incubation, more than 60% (10,000 ppm) of the initial amount of sucrose was consumed and significant concentrations of lactate and acetate were observed (10,320 ppm and 333 ppm, respectively). The amount of sucrose, glucose and fructose remaining after two days of incubation was 6009 ppm, 3603 ppm and 1302 ppm, respectively. The pH of the culture medium, initially at 7.0, decreased to 4.

These data indicate that probiotic species can be grown on maple sap when supplemented with a substrate rich in nitrogen, such as soy drink.

### Materials and Methods for Examples 2 and 3

Bacterial strains: Mixed culture BioK+ containing L. acidophilus and L. casei is commercially available from Bio-K Plus (Laval, Quebec, Canada). L. acidophilus was isolated from BioK+. L. rhamnosus was isolated from the commercial white cheese Damablanc (Damafro, St-Damase, Quebec, Canada). Lactobacillus acidophilus R0240 and Lactobacillus helveticus R0052 were provided by Dr. Thomas Tompkins (Lallemand Inc., Montreal, Quebec, Canada).

Media: Maple sap was obtained in March 2007 from the Centre d'Expérimentation et de Transfert Technologique Acéricole (CETTA, Pohénégamook, Quebec). The maple sap contained 23 g/L of sucrose and the pH was neutral. For Examples 2 and 3 below, maple sap (diluted at 20 g/L) was supplemented with yeast extract (5 g/L), Oxoid™ veggietone pea (20 g/L) (a nitrogen source), K₂HPO₄ (2 g/L), MnSO₄ (0.2 g/L) and MgSO₄ (0.05 g/L). A 20 g/L commercial sucrose-based medium, was similarly prepared.

Growth Conditions: Strains were first revived into Oxoid™ MRS medium overnight. Then, they were pre-cultured overnight in either the maple sap-based or the sucrose-based medium. This latter pre-culture was used to inoculate 20 mL of maple sap or sucrose-based medium. Cultures were incubated at 37° C. under static conditions in a 20 ml vial.

Analysis: During the course of the incubation, samples were taken for the analysis/determination of: optical density at 600 nm (OD₆₀₀ₙₘ), viable counts (colony forming unit (cfu) method), pH, lactic acid concentration (HPLC) and sucrose concentration (HPLC).

### EXAMPLE 2

### Growth in Maple Sap-based Medium

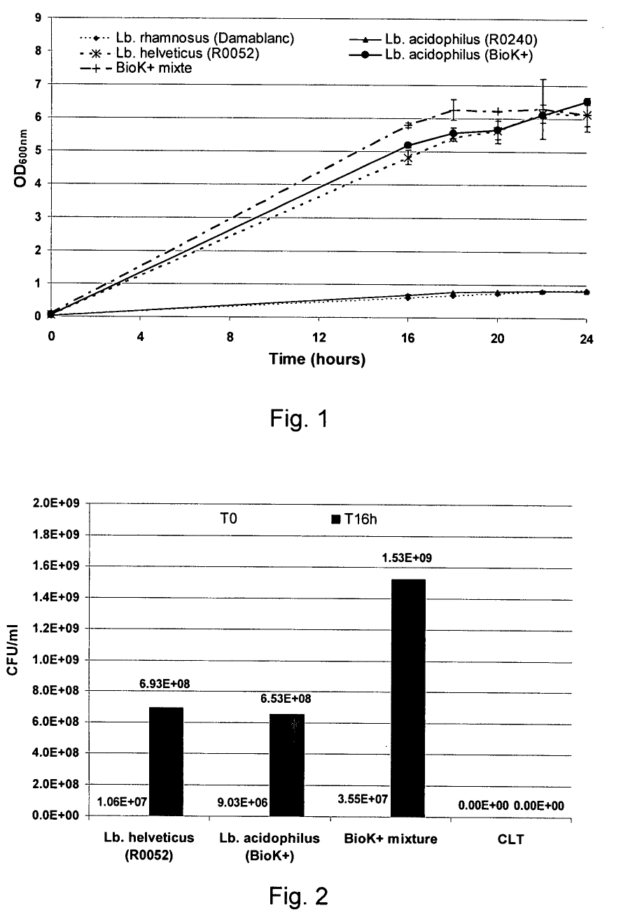

As shown in FIG. 1, the maple sap-based culture medium composed of maple sap from CETTA, veggietones pea, yeast extract, K₂HPO₄, MnSO₄ and MgSO₄ supported good growth (final OD₆₀₀ₙₘ around 6.0 reached between 18 and 20 hours of incubation) of three of the five strains tested. Based on these results, the viable counts were determined for the three best growers (L. helveticus R0052, L. acidophilus from BioK+, and the commercial mixed culture of BioK+).

FIG. 2 shows the cfu counts obtained after 16 hours of incubation in the maple sap-based medium. While L. acidophilus from BioK+ and L. helveticus R0052 grew to 6.0×10⁸ cfu/mL, the BioK+ mixture grew to 1.5×10⁹ cfu/mL. This latter cell concentration represents the targeted concentration for industrial production. CLT is a control with no sugar source.

### EXAMPLE 3

### Comparison of Maple Sap-based and Sucrose-based Media

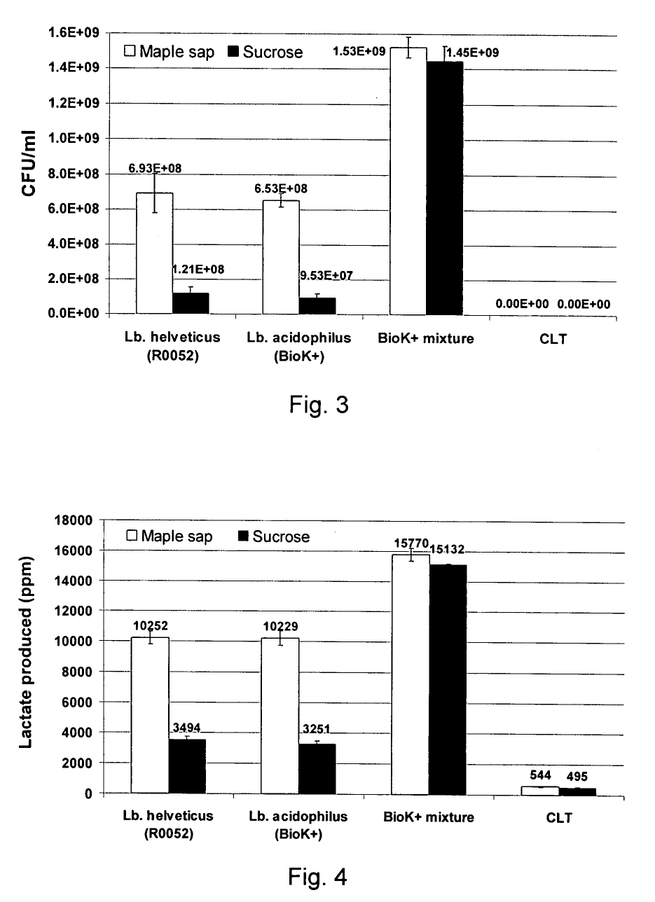

FIG. 3 shows that the use of maple sap-based medium improves production of probiotic bacteria in comparison to sucrose-based medium. In particular, the viable counts of L. acidophilus from BioK+ and L. helveticus R0052 were 5 times higher when maple sap was used as a basis for the preparation of the culture medium. The media were composed of the respective sugar sources together with veggietones pea, yeast extract, K₂HPO₄, MnSO₄ and MgSO₄. CLT is a control with no sugar source.

This “maple effect” was also observed in the production of lactic acid, as shown in FIG. 4. BioK+ mixture was the best producer with 15 g/L of lactic acid after 16 hours of fermentation. The production of lactic add by the two other strains (L. acidophilus from BioK+ and L. helveticus R0052) was around 3.5 g/L in the sucrose-based medium. However, the production was more than two-fold higher (10 g/L) after fermentation in the maple sap-based medium (FIG. 4).

References:
Woodward J.,and On M. 1998. Enzymatic Conversion of Sucrose to Hydrogen. Biotechnology Progress, 14 (6), 897-902.
Morin, A., Heckert, M., Poitras, E., Leblanc, D., Brion, F., and Moresoli, C. 1995. Exopolysaccharide production on low-grade maple sap by Enterobacter agglomerans grown in small scale bioreactors. Journal of Applied Bacteriology 79:30-37.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A method of growing probiotic bacteria comprising contacting the probiotic bacteria with maple sap, down-graded maple syrup or a mixture thereof.

2. The method according to claim 1, wherein the probiotic bacteria comprise bacteria from genus Lactobacillus.

3. The method according to claim 2, wherein the probiotic bacteria have a growth yield at least 1.5 times greater than growth yield on a sucrose-based medium.

4. The method according to claim 2, wherein the probiotic bacteria have a growth yield at least 2 times greater than growth yield on a sucrose-based medium.

5. The method according to claim 2, wherein the probiotic bacteria have a growth yield at least 4 times greater than growth yield on a sucrose-based medium.

6. The method according to claim 2, wherein the probiotic bacteria have a growth yield 2 to 4 times greater than growth yield on a sucrose-based medium.

7. The method according to claim 3, wherein the bacteria comprise Lactobacillus acidophilus or Lactobacillus helveticus.

8. The method according to claim 3, wherein the maple sap, down-graded maple syrup or mixture thereof is formulated into a medium comprising one or more growth supplements for the bacteria.

9. The method according to claim 8, wherein the growth supplement comprises a nitrogen source usable by the bacteria.

10. The method according to claim 9, wherein the nitrogen source comprises vegetal or animal protein.

11. The method according to claim 9, wherein the nitrogen source comprises vegetal protein.

12. The method according to claim 3, wherein the bacteria are incubated in the maple sap, down-graded maple syrup or mixture thereof at a temperature in a range from about 30° C. to about 45° C.

13. The method according to claim 3, wherein the bacteria are incubated in the maple sap, down-graded maple syrup or mixture thereof in a bioreactor.

14. A method of producing lactic acid comprising contacting probiotic Lactobacillus bacteria with maple sap, down-graded maple syrup or a mixture thereof.

15. The method according to claim 14, wherein the lactic acid is produced in an amount at least 1.5 times greater than in a sucrose-based medium.

16. The method according to claim 14, wherein the lactic acid is produced in an amount at least 2 times greater than in a sucrose-based medium.

17. The method according to claim 15, wherein the bacteria comprise Lactobacillus acidophilus, Lactobacillus casei, Lactobacillus helveticus or any mixture thereof.

18. The method according to claim 15, wherein the maple sap, down-graded maple syrup or mixture thereof is formulated into a medium comprising one or more growth supplements for the bacteria.

19. The method according to claim 18, wherein the growth supplement comprises a nitrogen source usable by the bacteria.

20. The method according to claim 19, wherein the nitrogen source comprises vegetal or animal protein.

21. The method according to claim 19, wherein the nitrogen source comprises vegetal protein.

22. The method according to claim 15, wherein the bacteria are incubated in the maple sap, down-graded maple syrup or mixture thereof at a temperature in a range from about 30° C. to about 45° C.

23. The method according to claim 15, wherein the bacteria are incubated in the maple sap, down-graded maple syrup or mixture thereof in a bioreactor.
